(19) United States
(12) Patent Application Publication
(10) Pub. No.: US 2025/0065093 A1
(43) Pub. Date: Feb. 27, 2025

(54) DRUG DELIVERY NEEDLE WITH ENHANCED PENETRATION CAPABILITY

(71) Applicant: Alma Therapeutics Ltd., Petach Tikva (IL)

(72) Inventor: Oz Cabiri, Hod HaSharon (IL)

(73) Assignee: Alma Therapeutics Ltd., Petach Tikva (IL)

(21) Appl. No.: 18/454,947

(22) Filed: Aug. 24, 2023

(51) Int. Cl. A61M 37/00

(52) U.S. Cl. CPC A61M 37/0015; A61M 2037/0023; A61M 2202/0007

(57) ABSTRACT

A drug delivery needle includes a body portion from which extends a penetration end that includes at least two sharp distal tips, the sharp distal tips being separated from each other by an arcuate trough that includes a cutting edge at least at a middle portion of the arcuate trough. At least the two sharp tips are made of a drug or include a drug therein or are coated with a drug. The drug is biodegradable.

### FIELD OF THE INVENTION

The present invention relates generally to drug delivery needles, and particularly to a drug delivery needle with enhanced penetration capability.

### BACKGROUND OF THE INVENTION

Drug delivery into mucosal tissue presents many challenges. In many places in the body, mucosal tissue includes three basic layers: the epithelium (the outermost layer), lamina propria, and the deeper sub-mucosal tissue. The lamina propria is rich in macrophages and lymphoid cells, and forms part of the barrier that protects internal tissues from external pathogenic microorganisms.

The ability for a drug to pass the epithelium and reach the lamina propria and the sub-mucosal tissue is a challenge. In particular, drug delivery by needle penetration into the mucosa has many challenges. For example, the mucosa in the gastrointestinal (GI) tract is covered with a continuously renewed viscous fluid which prevents needle penetration at many different angles of the needle tip with respect to the tissue. Another problem is the resilience to the mucosa to needle penetration; there is generally no counterforce to the needle penetration. The force of the needle tip trying to penetrate the mucosa causes the mucosa to be pushed in the direction of the force without the needle tip succeeding to penetrate the mucosa.

### SUMMARY

The present invention seeks to provide a drug delivery needle with enhanced penetration capability, as described in detail below.

There is provided in accordance with a non-limiting embodiment of the invention a drug delivery needle that includes a body portion from which extends a penetration end that includes at least two sharp distal tips, the sharp distal tips being separated from each other by an arcuate trough that includes a cutting edge at least at a middle portion of the arcuate trough, wherein at least the two sharp tips are made of a drug or include a drug therein or are coated with a drug, the drug being biodegradable. The sharp distal tips may be biodegradable.

Forked or Franseen hollow needles are known for use as biopsy needles. However, the structure of biopsy needles is such that the biopsy needle cuts out a portion of the tissue, detaches the cut portion and removes it for testing the cut specimen. There is an additional action of completing the cut by turning the needle or completing the cut by some other means.

In contrast, the structure of the drug delivery needle of the invention does not cut out any tissue and does not do any coring to the tissue. Instead, the tips of the drug delivery needle of the invention spread out and/or clear an area on the tissue for the cutting edge to pierce the tissue just enough for the drug to be delivered into the tissue. The tissue portion that is spread and/or cleared by the inventive needle is instead cut out by the prior art biopsy needle.

The cutting edge of the inventive needle cuts the tissue in one movement of penetration, that is, makes an incision in the tissue, and pushes the soluble drug into the incision instead of into a stretched hole. This reduces the tissue's resistance to being penetrated. The needle tips touch the tissue on the sides of the penetrated area to keep the penetrated area taut; this provides a counter force that allows the cutting edge to cut the tissue. The needle can now remain in the tissue for the required time for the drug to dissolve into the tissue (without limitation, for up to an hour or so). The drug is inserted directly into the target layer and the needle reduces the phenomenon of local crushing, which in the prior art leaves the epithelial cells intact and prevents the introduction of the drug molecule.

### BRIEF DESCRIPTION OF DRAWINGS

The present invention will be understood and appreciated more fully from the following detailed description, taken in conjunction with the drawings in which:

FIG. 1A is a simplified perspective illustration of a drug delivery needle, in accordance with a non-limiting embodiment of the invention.

FIGS. 1B and 1C are simplified illustrations of the drug delivery needle from two other views.

FIG. 2 is a simplified perspective illustration of an array of the drug delivery needles, in accordance with a non-limiting embodiment of the invention.

FIG. 3A is a side-view illustration of the array of the drug delivery needles.

FIG. 3B is a sectional view of the array of the drug delivery needles, taken along lines 3B-3B in FIG. 2.

FIG. 3C is a sectional view of the array of the drug delivery needles, taken along lines 3C-3C in FIG. 2.

### DETAILED DESCRIPTION

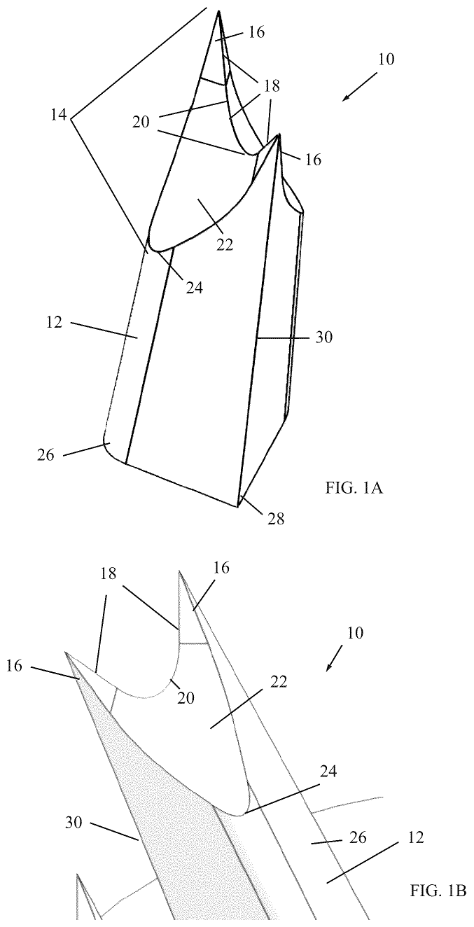

Reference is now made to FIGS. 1A-1C, which illustrate a drug delivery needle 10, in accordance with a non-limiting embodiment of the invention. As used throughout the description and claims, the term needle encompasses any sharp penetration member.

Drug delivery needle 10 may include a body portion 12 from which extends a penetration end 14 that includes at least two sharp distal tips 16. The tips 16 are separated from each other by an arcuate trough 18 that includes a cutting edge 20 at least at a middle portion of the trough 18 (the area between the two leader lines of numeral 20 in FIG. 1A). The cutting edge 20 may extend completely along the entire arcuate length of arcuate trough 18, that is, from one tip 16 to the next tip 16. The arcuate trough 18 may have the shape of a parabola or other conical section or other arcuate shapes.

The body portion 12 of needle 10 may be formed with a bevel 22 that extends from arcuate trough 18 to a heel 24. The bevel 22 may be arcuate, such as being concave, that is, having an arcuate depression with respect to arcuate trough 18 and heel 24.

The outer perimeter of body portion 12 of needle 10 may be round or may have edges. For example, in the non-limiting illustrated embodiment, body portion 12 has at least one rounded corner 26, and at least one non-rounded corner 28. An edge 30 of the non-rounded corner 28 may extend up to the distal tip 16.

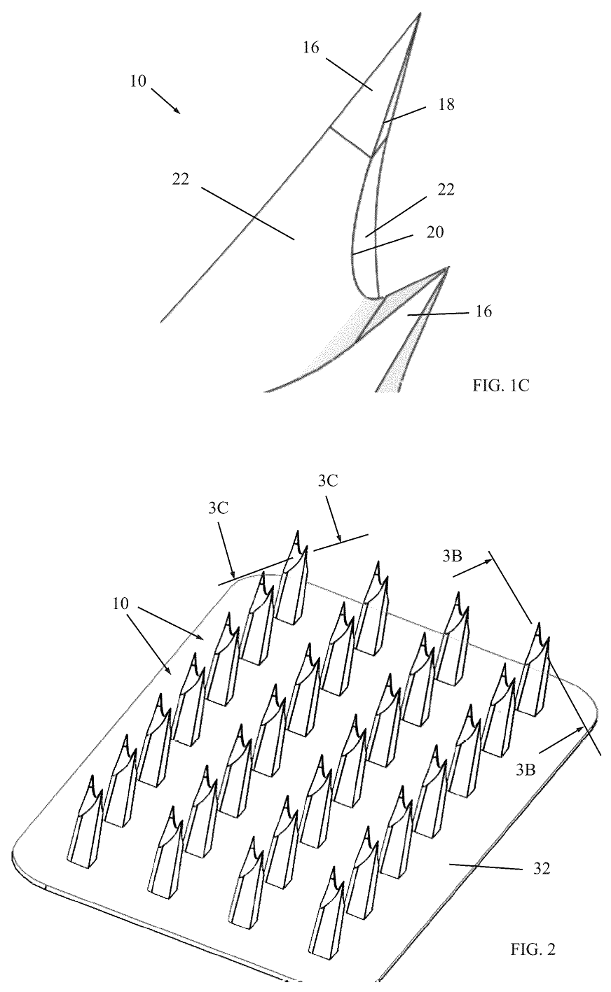

FIG. 2 illustrates an array of the drug delivery needles 10 mounted on a substrate 32, which may or may not be pliable, in accordance with a non-limiting embodiment of the invention.

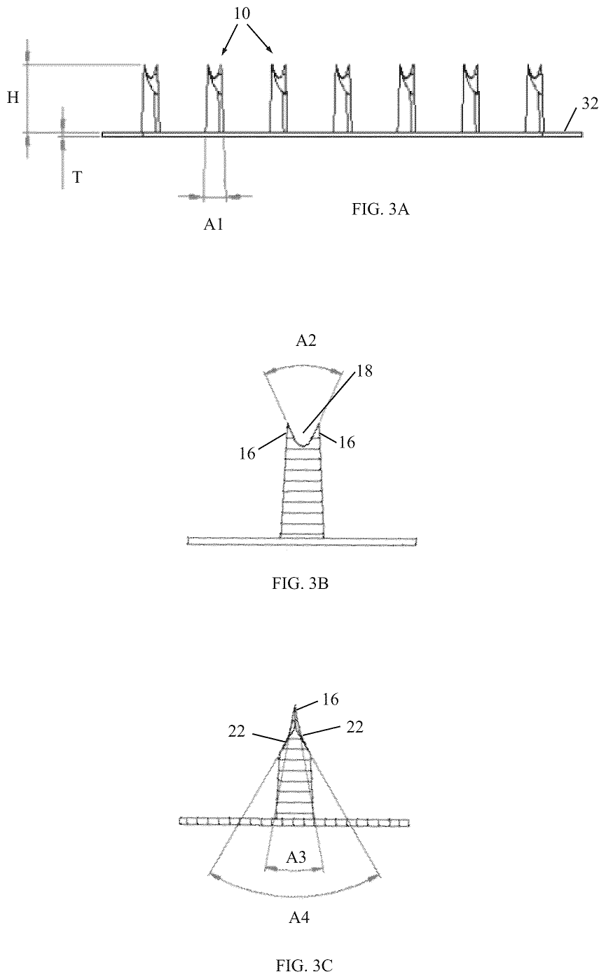

As seen in FIG. 3A, the drug delivery needle 10 may have a height H, which may be, without limitation, in the range of 1.2-2.0 mm. The substrate 32 may have a thickness T, which may be, without limitation, in the range of 0.05-1.5 mm. Needle 10 may taper slightly towards the distal tip at an angle A1, which may be, without limitation, in the range of 2-6°. The distal tips 16 may have equal heights or unequal heights.

As seen in FIG. 3B, the arcuate trough 18 may subtend an angle A2, which may be, without limitation, in the range of 30-70°.

As seen in FIG. 3C, the tip 16 may subtend an angle A3, which may be, without limitation, in the range of 10-30°. The bevel 22 may subtend an angle A4, which may be, without limitation, in the range of 40-80°.

The needle 10 or at least the tip 16 may include a drug as described in PCT Patent Application WO 2023/031887. As used herein, “drug”, “active agent”, “therapeutic” or “medicine” may be used interchangeably and includes small molecules, biologies, diagnostics, or active pharmaceutical ingredients used to treat or diagnose a patient. They can refer to any agent that, when administered, has a therapeutic effect and/or elicits a desired biological and/or pharmacological effect. Generally, an active agent may refer to more than one active agent. Examples of active agents that may be suitable for use include but are not limited to chemotherapeutic agent, interferon, an antibody, an antibiotic, growth hormone, parathyroid hormone, a glucose regulating agent, an insulin compound, an incretin hormone, a GLP-1 compound or exenatide, antiviral, protease inhibitor or an anti-seizure compound or other active agents which would otherwise chemically degrade or have limited permeability if released within the intestine lumen of the gastrointestinal tract. In various embodiments of any of the aspects presented, the active agent is a peptide sequence, protein, an enzyme, a polysaccharide, or a polynucleotide, amino acids, nucleotides, carbohydrates, sugars, lipids, nucleoproteins, glycoproteins, lipoproteins, steroids, etc. whether naturally occurring or artificially created (e.g., by synthetic or recombinant methods) that are commonly found in cells and tissues. Specific classes of biologies include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA. In some embodiments of any of the aspects presented, the active agent is in an amount of the active agent to produce a desired therapeutic effect is less than an amount to produce a corresponding effect if the agent was orally delivered without enclosure in the ingestible formulation. In some embodiments of any of the aspects presented, the active agent is a combination of active agents. In some embodiments of any of the aspects presented, the active agent would chemically degrade, have limited permeability, or impose a deleterious effect on the subject if released within the lumen of the gastrointestinal tract. In some embodiments of any of the aspects presented, the active agent comprises a polypeptide that is chemically degraded or have limited permeability in the GI tract and the agent is delivered into the wall of the small intestine with minimal or no loss in binding affinity or specificity to a target binding site.

As described in PCT Patent Application WO 2023/031887, needle 10 or at least the tip 16 may contain therein a drug or the drug may be a solid which is shaped as the needle or at least top 16. The needle 10 or at least the tip 16 may be biodegradable, so that after penetrating the mucosa the drug and the needle or tip are absorbed in the mucosal tissue.

Accordingly, needle 10 may be a penetrating member which is a tissue solid controlled-release penetrating member that includes active agent loaded microparticles contained or shaped as a microneedle. In some embodiments, the penetrating member comprises a first molding material. In some embodiments, the penetrating member comprises a mixture of a) a polymer first molding material and b) a second molding material comprising particles (e.g., rigid particles or microparticles) dispersed therein, wherein the first molding material is micro molded into the plurality of microneedles 10. In some embodiments, the penetrating member comprises an enteric coating or layer and a molding material comprising rigid particles dispersed therein, wherein the enteric coating is placed in the mold and a molding material with rigid particles are micro molded into the plurality of microneedles 10.

In various embodiments, the solid controlled-release penetrating member further comprises an outer coating of intestine environment protective component. In various embodiments, the intestine environment protective component surrounds or coats the controlled release coating. In various embodiments, the tip is at least partially coated, or the tip is formulated as a separate but adjacent and distal extension of the active agent formulation. In various embodiments, the solid controlled-release penetrating member comprises an intestine environment protective component and a tip coating. In some embodiments, the active agent is specifically released in the general area of the interstitial space of the intestine due to the choice of intestine environment protective component which may also target the pH specific to the interstitial space.

In various embodiments, the controlled release formulation includes an active agent and controlled release component. In some embodiments, the solid controlled-release penetrating member comprising the active agent is i. a mixture of active agent and controlled release component; or ii. an immediate release active agent inner core and controlled release coating.

The control release coating may include pH sensitive (e.g., cationic acrylic/methacrylic copolymer or acrylic/methacrylic copolymer having a pH threshold at about 6.5) or sustained release polymers or excipients.

Sustained release polymers include for example lipophilic coatings. Lipophilic coatings may include an excipient selected from the list consisting of: polyglycolide (PGA), polylactide (PLA), poly-epsilon-caprolactone, poly dioxanone (a polyether-ester), poly lactide-co-glycolide, polyamide esters, polyalkalene esters, polyvinyl esters, polyvinyl alcohol, and polyanhydrides.

Typically, in various embodiments, coatings mentioned throughout, are located in a layer externally located relative to the active agent inner (e.g., inner core), and form the tip coating, controlled release coating or intestine environment protective component. Coatings may have a width of between 2 and 25 μm and preferably less than 10 μm. In addition, one may measure a combined thickness of multiple layers of coatings, for example, the outer control release coating and intestine environment protective component and the combined thickness may be less than 50 pm.

In some embodiments, the immediate release active agent inner core can be a hollow shell with dry active agent, or an active agent admixed with a biodegradable excipient or polymer. For example, immediate release active agent inner core may include any one or more biodegradable polymers having monomer units designed for drug delivery. Relevant examples of biodegradable polymers include but are not limited to: natural polymers; polyesters including poly(lactic acid) (“PLA”), poly(lactic-co-glycolic acid) (“PLGA”), poly(caprolactone) (“PCL”), poly(glycolic acid) (“PGA”); chitosan; poly(ortho esters) such as (POE I, POE II, POE III or POE IV); hyaluronic acid; poly(anhydrides); poly(amides); poly(ester amides); poly(phosphoesters); poly(alkyl cyanoacrylates) (“PACA”). Additional excipients for immediate release of an active agent are also possible and can include for example dextrin, polysaccharides, arginine, carboxymethylcellulose, water miscible polymers including polyethylene glycol (“PEG”), polyvinyl alcohol (“PVA”) or polyvinylpyrrolidone (“PVP”) (optionally in combination with polymers containing a cationic acrylic/methacrylic copolymers, said polymers being based on methacrylic acid esters and a small portion of trimethyl aminoethyl methacrylate chloride' [such as, Eudragit RS 100®] or based on meth acryl acid esters and a small portion of amino methacrylate copolymers [such as Eudragit E 100®]. Other options are also possible and can be chosen by those skilled in the art.

The active agent inner core can further comprise a stabilizer, buffer, polymer, antioxidant, diluent, lubricant, binder, or plasticizer and can be chosen by those skilled in the art. Surfactant can also be added and can be especially important to contribute to structural stability and reduce exposure of hydrophobic regions so as to decrease or limit interface-induced aggregation and protein-protein interaction and resulting mechanical stress. Examples include poloxamers, polysorbate 20 and especially polysorbate 80. Relevant ratios to active agent may depend on the active agent and can be calculated.

In some embodiments, the mixture comprises a controlled release component such as a non-disintegrating carrier matrix selected from the list consisting of acacia, sodium alginate, gelatin, carboxymethyl cellulose sodium, methylcellulose, ethylcellulose, cellulose acetate or polyacrylates (e.g., ammonio methacrylate copolymers i.e., Eudragit RS/RL), polyethylene glycol (PEG), polyvinyl polymers, methylcellulose (e.g., HPMC, HPC) and ethylcellulose.

In another embodiment, the mixture comprises a controlled release component selected from the list consisting of polyvinyl polymers, methylcellulose and ethylcellulose. The ethyl cellulose may be a low-viscosity ethyl cellulose. When a low-viscosity ethyl cellulose is used, the mixture further comprises a pore-forming agent selected from the group consisting of hydroxypropyl cellulose and hydroxypropyl methylcellulose. The matrix may additionally include further excipients such as binders, fillers, process enhancers such as lubricants and glidants. In some embodiments, the excipients do not include a disintegrant.

In some embodiments of any of the aspects presented, the one or more solid controlled-release penetrating member are at least partially covered with polymer and specifically a slow-release polymer which delays exposure of the penetrating member. Examples include a pH-dependent enteric coating having sensitivity at pH greater than 6.0, PLGA, PLGA/PEG, PLGA/PAA, an aqueous pore forming film (water soluble ethyl cellulose and water soluble hydroxypropyl cellulose), polymer or semi-permeable film (e.g., combination of PVP K30 and hydrophilic polyethylene glycol such as PEG 6000), silk fibroin, a lipophilic (PCL, PDO, HCO) or hydrophobic polymer. This coating may additionally include pore formers, plasticizers, and processing enhancing agents such as lubricants.

Suitable the pore formers are known in the art and include HPC. HPMC, polyethylene glycol, poloxamer, povidone or a saccharide. Preferably, it is HPC, HPMC, polyethylene glycol, or povidone. Suitable amounts of pore formers in the coating polymer to pore former of 5:1 to 2:1 or 5:1 to 1:1 and preferably ratios of 100:35 to 100:45.

In various embodiments, the solid controlled-release penetrating member includes an outer control release coating comprises any suitable control release coating forming polymers which enable diffusion-based controlled release known in the art for instance: polyacrylates such as ammonio methacrylate copolymers (Eudragit RS/RL), polyvinylacetate, hydroxypropyl methylcellulose (optionally with ethylcellulose), carboxymethylcellulose, hydroxypropyl cellulose, polyvinyl pyrrolidone (optionally with carboxymethylcellulose), alginates (e.g., sodium alginate or alginate/chitosan), methylcellulose, lipophillic poly-e-caprolactone and ethyl cellulose. In a preferred embodiment, the outer controlled release coating is ethylcellulose, cellulose acetate, or polyacrylates such as ammonio methacrylate copolymers (Eudragit® RS or RL), polyvinylacetate or combinations thereof. The coating further includes a plasticizer. The amount of plasticizer is typically between 8 and 35% or preferably 10 to 25% relative to the control release polymer.

In some embodiments, the control release coating comprises a low-viscosity ethyl cellulose, a pore-forming agent, and a plasticizer. The low-viscosity ethyl cellulose and a pore-forming agent may be present in a ratio of the pore-forming agent to low-viscosity ethyl cellulose of about 9:1 to about 1:1. The low-viscosity ethyl cellulose and a pore-forming agent may be present in a ratio of the poreforming agent to low-viscosity ethyl cellulose of about was 9:1 to 5:5 or 7:4 to 4:5. Relevant ratios include but are not limited to 9:1, 8:2, 6:4, 5:5 and 4:5. In some cases, control release coating having a low-viscosity ethyl cellulose may further comprise a plasticizer such as triethyl citrate.

In some embodiments, the control release coating comprises a hydroxypropyl cellulose or hydroxypropyl methylcellulose having a viscosity of about 4 cP to about 14 cP.

In some embodiments, the solid controlled-release penetrating member has an outer control release coating which is a sustained release or cationic acrylic/methacrylic copolymer.

What is claimed is:

1. A drug delivery needle comprising:
a body portion from which extends a penetration end that comprises at least two sharp distal tips, said sharp distal tips being separated from each other by an arcuate trough that comprises a cutting edge at least at a middle portion of said arcuate trough, wherein at least said two sharp distal tips are made of a drug or comprise a drug therein or are coated with a drug, said drug being biodegradable.

2. The drug delivery needle according to claim 1, wherein said at least said two sharp distal tips are biodegradable.

3. The drug delivery needle according to claim 1, wherein said cutting edge extends completely between said sharp distal tips.

4. The drug delivery needle according to claim 1, wherein said arcuate trough is shaped as a conical section.

5. The drug delivery needle according to claim 1, wherein said body portion is formed with a bevel that extends from said arcuate trough to a heel.

6. The drug delivery needle according to claim 5, wherein said bevel is concave, having an arcuate depression with respect to said arcuate trough and said heel.

7. The drug delivery needle according to claim 1, wherein an outer perimeter of said body portion has at least one rounded corner and at least one non-rounded corner.

8. The drug delivery needle according to claim 7, wherein an edge of said non-rounded corner extends up to one of said sharp distal tips.

9. The drug delivery needle according to claim 1, wherein a plurality of said drug delivery needles are mounted on a substrate.

10. The drug delivery needle according to claim 1, wherein said body portion tapers towards said sharp distal tips.

11. The drug delivery needle according to claim 1, wherein said sharp distal tips have equal heights.

12. The drug delivery needle according to claim 1, wherein said sharp distal tips have unequal heights.
